(19) United States
(12) Patent Application Publication
(10) Pub. No.: US 2010/0075270 A1
(43) Pub. Date: Mar. 25, 2010

(54) Angularly adjustable maxillary distractor and method of distraction

(76) Inventors: Alvaro A. Figueroa, Glencoe, IL (US); John Polley, River Forest, IL (US); Thomas Johnston, Jacksonville, FL (US)

Correspondence Address: ROGERS TOWERS, P.A., 1301 RIVERPLACE BOULEVARD, SUITE 1500, JACKSONVILLE, FL, 32207, US

(21) Appl. No.: 12/284,280

(22) Filed: Sep. 19, 2008

(51) Int. Cl. A61C 3/00; A61B 17/58; A61B 17/04

(52) U.S. Cl. 433/18; 606/105; 606/71

(57) ABSTRACT

A maxillary distractor assembly having a malar buttress plate member and a vertical arm member, wherein the malar buttress plate member is detachably connected to the vertical arm member such that the malar buttress plate member may be left in the patient when the remainder of the assembly is removed.

### BACKGROUND OF THE INVENTION

This invention relates generally to the field of medical compression and distraction devices in the field of bone fixation, repair and regeneration, and more particularly relates to such devices and methods utilized in maxillary craniofacial repair, facial reconstruction, and treatment for congenital, developmental and traumatic defects.

It is often desirable or necessary to effect reconstruction or repair of the craniofacial bones defining the face of a person, such as the midface, the mandible and/or the maxilla. This need may arise from congenital conditions, developmental disorders or trauma. In many circumstances the abnormalities are corrected by first performing an osteotomy, i.e., cutting or fracturing a bone to create two segments separated by a gap or space. In some instances the bone segments will be shifted into the proper orientation and alignment, and then fixed in place relative to each other until bone growth across the gap results in the formation of a unitary bone member. In other instances it is necessary to lengthen the original bone member, in which case devices known as distractors are utilized. A distractor is a device that has affixation means, such as bone plates, that are joined to each of the bone segments on opposite sides of the osteotomy. The device further includes distraction means that allows the distance between the bone plates to be slowly increased over time, thereby allowing new bone growth to occur between the bone segments. The new bone growth increases in dimension until the proper bone length is achieved, at which time the distraction process is halted and the distractor is removed.

As a typical example, it is often desired to advance the midface or maxillary region relative to the jaw and skull to correct for maxillary hypoplasia, where the upper lip and/or nose are depressed relative to the remainder of the face structure. In this case the osteotomy may be performed across the maxilla to the nasal cavity, and a pair of distractors are affixed across the osteotomy gap, or an external distraction apparatus is mounted to the skull with affixed means to distract the anterior maxillary segment. Gradual extension of the distractors in unison advances the anterior maxillary segment relative to the posterior maxillary segments while bone regeneration fills in the osteotomy gap. When the proper position is achieved, distraction is halted. After sufficient regeneration and healing, the distractors are removed.

The known protocols for distraction of the maxilla possess inherent problems. Affixation of the distractors involves invasive surgery, with multiple incisions and implantations of bone screws to affix the distractor bone plates being necessary. Removal of the bone plates after regeneration has occurred, particularly those bone plates affixed to the maxilla, require a new incision and extended healing time. It is desirable to reduce the number of invasive procedures that must be done to address the craniofacial anomalies, and in particular desirable to reduce or eliminate invasive procedures post-regeneration.

It is an object of this invention to provide a craniofacial maxillary distraction device and method that addresses the problems set forth above. This object as set forth above will be met as provided for in the disclosure to follow, and other objects not expressly set forth at this time will become apparent upon review of the disclosure to follow.

### SUMMARY OF THE INVENTION

The invention is an angularly adjustable maxillary distractor assembly and method of distraction to correct maxillary deficiencies, cleft palates and similar conditions. The angularly adjustable maxillary distractor assembly allows for both vertical and horizontal distraction, allows for adjustment of the distraction vector both prior to implantation and during distraction, and is easily removed from the patient without the need for a post-regeneration invasive procedure.

In general, the angularly adjustable maxillary distractor assembly comprises a malar buttress plate member detachably mounted to a vertical arm member, wherein the vertical arm member is length adjustable. The assembly further comprises operational means for distraction comprising a horizontal distractor assembly comprising a maxillary fixation plate member, wherein the maxillary fixation plate member is translatable along the longitudinal axis of the horizontal distractor assembly. The vertical arm member and the horizontal distractor assembly are joined by releasable angular adjustment means, such that the angle between the vertical arm member and the horizontal distractor assembly may be adjusted as required.

To effect distraction, an osteotomy is performed to separate the maxilla from the malar buttress and zygomatic buttress. A pair of angularly adjustable maxillary distractor assemblies is utilized, one for each side of the maxilla. The malar buttress plate member is secured to the malar buttress and the maxillary fixation plate is affixed to the maxilla, usually by wire or screw connection to an appliance attached to the upper teeth. The horizontal distractor assembly is then angularly adjusted relative to the vertical arm member for the proper distraction vector and the angle is fixed. Distraction is then effected by incrementally extending the maxillary fixation plate as osteogenesis fills in the osteotomy gap. During the distraction process, the distractor vector may be altered as necessary by adjusting the angle between the horizontal distractor assembly and the vertical arm member. When the process is completed, the horizontal distractor assembly is removed by disconnecting it from the vertical arm member, and the vertical arm member is removed by disconnecting it from the malar buttress plate member, which is left in the patient, whereby the soft tissue adjacent to the malar buttress plate is closed in normal fashion over the malar buttress plate or is simply allowed to naturally heal over the malar buttress plate.

### BRIEF DESCRIPTION OF THE DRAWINGS

FIG. 1 is a perspective view of an embodiment of the angularly adjustable maxillary distractor assembly.

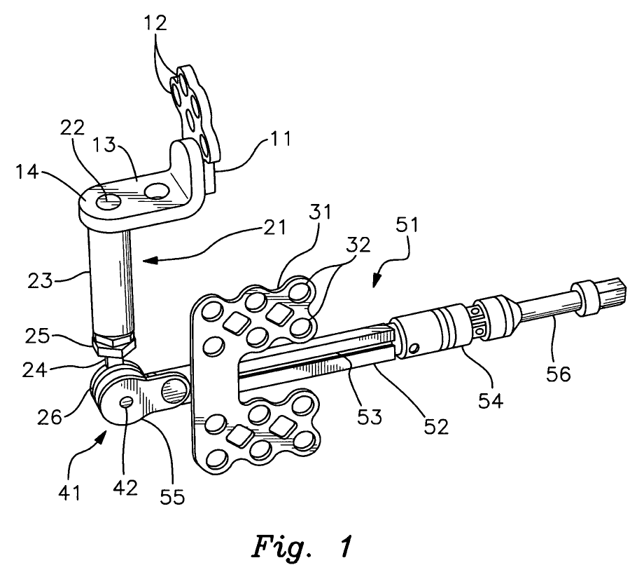

FIG. 2 is an alternate perspective view of the embodiment of FIG. 1.

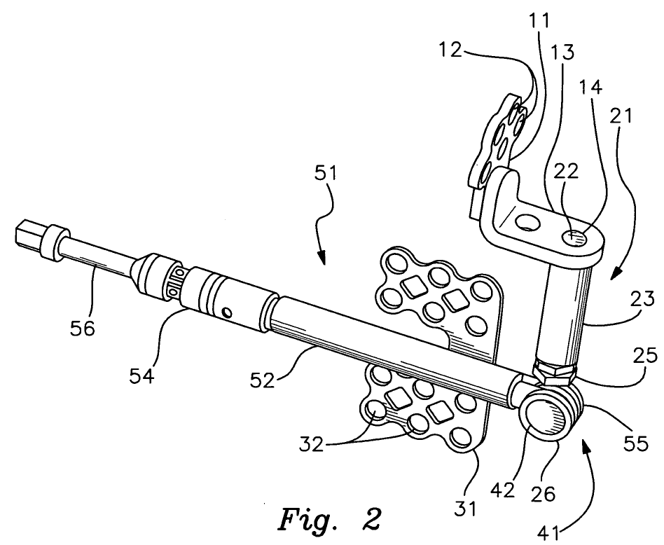

FIG. 3 is a side view of an alternative embodiment of the angularly adjustable maxillary distractor assembly, showing the malar buttress plate member rotated relative to the horizontal maxillary distractor assembly.

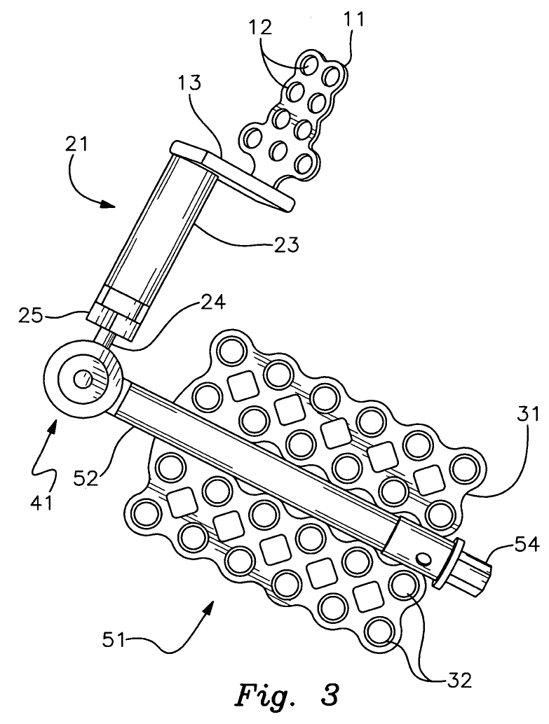

FIG. 4 is a view of embodiment of FIG. 3 as implanted on a patient.

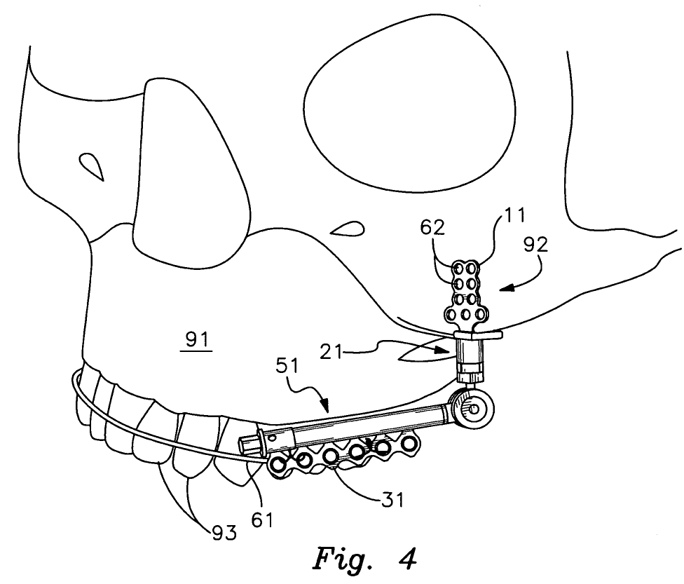

### DETAILED DESCRIPTION OF THE INVENTION

With reference to the drawings, the invention will now be described in detail with regard for the best mode and preferred embodiment. In general, the invention is an angularly adjustable maxillary distractor assembly and its related method of distraction, comprising means for attachment of the assembly to the malar buttress or adjacent bone structure, means for attachment of the assembly to the maxilla, means for distraction of the maxilla relative to the malar buttress, means for adjusting the angle of the displacement vector prior to and after implantation of the assembly in a patient, and means for detachably retaining a malar buttress plate such that the malar buttress plate is easily detached from the remainder of the distractor assembly when the corrective procedure is finished.

The angularly adjustable maxillary distractor assembly comprises means for attachment of the assembly to the malar buttress 92 comprising a detachable malar buttress plate member 11 and a length-adjustable vertical arm member 21, means for attachment of the assembly to the maxilla 91 comprising a maxillary fixation plate member 31, means for distraction of the maxilla 91 relative to the malar buttress 92 comprising a horizontal distraction assembly 51, and means for connecting the vertical arm member 21 to the horizontal distraction assembly 51 in a manner that allows for adjusting the angle of the displacement vector prior to and after implantation of the assembly in a patient comprising a releasable, adjustable joint assembly 41. The vertical arm member has a longitudinal axis defining a fixed axis, and the horizontal distraction assembly 51 has a longitudinal axis defining a distraction vector, i.e. the direction of movement of the maxilla 91 relative to the malar buttress 92 during the distraction process. The angle of the displacement vector is defined to be the angle formed between the fixed axis of the vertical arm member 21 and the longitudinal axis of the horizontal distraction assembly 51.

The detachable malar buttress plate member 11 comprises a plurality of screw-receiving apertures 12. The malar buttress plate member 11 may be presented initially as a generally planar member having any peripheral configuration and is preferably composed of a malleable material such that the shape of the malar buttress plate member 11 may be adjusted by bending or cutting to conform to the contours of the malar buttress 92. The malar buttress plate member 11 is adapted to be attachable to the malar buttress 92 in known manner using bone screws 62. The malar buttress plate member 11 and bone screws 62 may be composed of permanent or bioresorbable material. A mounting flange 13 is provided as an extension of the malar buttress plate member 11 or may be a separate member joined to the malar buttress plate member 11. As shown in the drawings, the mounting flange 13 may be disposed at a right angle to the malar buttress plate member 11. The mounting flange 13 is provided with a means for connecting the mounting flange 13 to the vertical arm member 21, which as shown for example comprises mechanical interlocking means such as a threaded aperture 14 to receive the threaded post member 22 of the vertical arm member 21.

The vertical arm member 21 is length adjustable, and as shown comprises an internally threaded sleeve member 23 mounted onto a slotted, externally threaded rod member 24, such that relative rotational movement of the sleeve member 23 on the rod member 24 results in a lengthening or shortening of the vertical arm member 21 along its longitudinal axis. A locking nut member 25 is provided to secure the vertical arm member 21 at the desired length. In this manner the distance between the malar buttress plate member 11 and the horizontal distraction assembly 51 may be adjusted as required for a particular patient and condition. While the mechanism as described is suitable for operation of the length adjustable vertical arm member 21, other mechanisms of similar function may be utilized. The post member 22 for releasable connection to the malar buttress plate member 11 is disposed on the upper end of the vertical arm member 21, and the lower end of the vertical arm member 21 comprises a component of the releasable joint assembly 41, which as shown for example comprises an apertured pivot flange 26 that receives a set screw 42. This overall structure also allows the malar buttress plate member 11 to be rotated relative to the joint assembly 41 for proper location of the distraction vector upon implantation.

The means for distraction of the maxilla relative to the malar buttress may comprise any of the known operational distraction mechanisms that define a distraction vector, but as shown a suitable mechanism comprises a horizontal distractor assembly 51 comprising an elongated sleeve housing 52 retaining a threaded rod member 53 in a manner that allows for rotation of the rod member 53 within the sleeve housing 52, the rod member 53 connected to a drive member 54 on the free end of the assembly 51 that receives an activation arm member 56. The distractor assembly 51 further comprises a maxillary fixation plate member 31 that is connected to the rod member 53 in known manner that allows the maxillary fixation plate member 31 to advance or retract longitudinally when the rod member 53 is rotated. For example, the maxillary fixation plate member 31 may be provided with an internally threaded aperture or bore that receives the threaded rod member 53. The maxillary fixation plate member 31 is the means for connecting the horizontal distraction assembly 51 to the maxilla 91, and as shown may comprise apertures 32 for receiving a rod, wire or screw, such as would be used to connect the maxillary fixation plate member 31 to a maxilla appliance member 61 of known type affixed to the upper teeth 93 of the patient. The maxillary fixation plate member 31 may be composed of a malleable material to allow it to be shaped or cut as required, and the peripheral configuration, location of the apertures, etc. may vary as required for best connecting the maxillary fixation plate member 31 to the maxilla appliance member 61. Opposite to the free end of the horizontal distractor assembly 51 is a component of the releasable joint assembly 41, which as shown comprises an internally threaded, apertured pivot flange member 55 which when properly oriented in a face-to-face manner with the vertical arm pivot flange 26 receives the threaded end of set screw 42, such that the relative positions of the vertical arm member 21 and the horizontal distractor assembly 51 can be altered and then fixed in place by loosening and tightening the set screw 42. The relative positions of the vertical arm member 21 and the horizontal distractor assembly 51, and more particularly the longitudinal axis of the vertical arm member 21 and the distraction vector defined by the longitudinal axis of the horizontal distractor assembly 51, defines the distraction angle of the adjustable maxillary distractor. In this manner, as more fully set forth below, the distraction angle can be adjusted prior to implantation of the adjustable maxillary distractor, and more importantly, the distraction angle and distraction vector can be adjusted after implantation of the adjustable maxillary distractor and during the distraction process.

The method of distraction utilizing the angularly adjustable maxillary distractor comprises assembling the detachable malar buttress plate member 11 and the vertical arm member 21. The location of the osteotomy is determined and marked. The malar buttress plate member 11 is contoured or sized as required to conform to the anatomy of the malar buttress 92 at the location of fixation., and the malar buttess plate member 11 is temporarily mounted onto the malar buttress 92 with the vertical arm member 21 perpendicular to the occlusal plane. The horizontal distraction assembly 51 is then connected to the vertical arm member 21 using the set screw 42 of the releasable joint assembly 41. The height of the vertical arm member 21 is adjusted relative to the occlusal plane to properly position the horizontal distraction assembly 51, and the entire assembly is removed. The maxillary fixton plate 31 is then contoured or cut as required. This operation is performed for both of the angularly adjustable maxillary distractors. The maxilla appliance member 61 may now be attached to the teeth 93. The osteotomy is performed to separate the maxilla 91 from the malar buttress 92 and the malar buttress plate members 11 are now permanently mounted to the malar buttress 92. The proper distraction vector is now determined and the distraction angle between the horizontal distractor assembly 51 and the vertical arm member 21 is adjusted as required. The maxilla fixation plate members 31 are now connected to the maxilla appliance member 61. Once the latency period has passed, distraction procedure begins. The activation arm 56 is rotated so as to extend the maxilla fixation plate member 31 a short distance, such as for example 1.0 m, which causes the maxilla 91 to be moved from the malar bone an equivalent distance. As bone regenerates in the osteotomy gap, the maxilla 91 is progressively extended until the desired orientation is achieved. During the distraction process it may be necessary to adjust the distraction vector, which is easily accomplished by adjusting the distraction angle. The set screw 42 of the joint assembly 41 is loosened, the horizontal distractor assembly 51 is pivoted as required relative to the vertical arm member 21, and the set screw 42 is tightened. Once proper distraction is achieved, the device is allowed to remain in place for a period of 8 to 12 weeks, although the activation arm 56 may be removed at this time since adjustment is no longer necessary.

The structure of the adjustable maxillary distractor as disclosed above further allows for easy removal of the majority of the components when the distraction procedure has been completed. To accomplish this, the maxillary fixation plate member 31 is released from the maxilla appliance member 61, such as by cutting the wire or rod or removing any screws. The set screw 42 of the releasable joint assembly 41 is removed or at least withdrawn from the horizontal distractor assembly pivot flange 55, such that the horizontal distractor assembly 51 is no longer connected to the vertical arm member 21 and can be removed. The vertical arm member 21 is then disconnected from the malar buttress plate member 11 by unthreading the post member 22. The malar buttress plate member 11 remains affixed to the patient.

It is contemplated that equivalents and substitutions for elements set forth above may be obvious to those of ordinary skill in the art, and therefore the true scope and definition of the invention is to be asset forth in the following claims.

We claim:

1. A maxillary distractor assembly comprising means for attachment of the assembly to the malar buttress of a patient, said attachment means comprising a malar buttress plate member and a vertical arm member, and wherein said malar buttress plate member is detachably connected to said vertical arm member.

2. The assembly of claim 1, wherein said malar buttress plate member comprises a mounting flange member, and wherein said malar buttress plate member is detachably connected to said vertical arm member by said mounting flange.

3. The assembly of claim 1, wherein said malar buttress plate member is detachably connected to said vertical arm member using mechanical interlocking means.

4. The assembly of claim 3, wherein said mechanical interlocking means comprise a threaded aperture and a threaded post.

5. The assembly of claim 2, wherein said malar buttress plate member is detachably connected to said vertical arm member using mechanical interlocking means.

6. The assembly of claim 5, wherein said mechanical interlocking means comprise a threaded aperture and a threaded post.

7. An angularly adjustable maxillary distractor assembly comprising:
a horizontal distractor assembly pivotally and detachably connected to a vertical arm member whereby the angle between said horizontal distractor assembly and said vertical arm member is adjustable; and
a malar buttress plate member detachably connected to said vertical arm member;
whereby with said malar buttress plate member connected to said vertical arm member, said horizontal distractor assembly is detachable from said vertical arm member.

8. The maxillary distractor assembly of claim 7, wherein said malar buttress plate member comprises a mounting flange member, and wherein said malar buttress plate member is detachably connected to said vertical arm member by said mounting flange.

9. The maxillary distractor assembly of claim 7, wherein said malar buttress plate member is detachably connected to said vertical arm member using mechanical interlocking means.

10. The maxillary distractor assembly of claim 9, wherein said mechanical interlocking means comprise a threaded aperture and a threaded post.

11. The maxillary distractor assembly of claim 8, wherein said malar buttress plate member is detachably connected to said vertical arm member using mechanical interlocking means.

12. The assembly of claim 11, wherein said mechanical interlocking means comprise a threaded aperture and a threaded post.

13. A method of correcting a deformity in a human maxilla through a distraction process comprising:
creating two osteotomies in the maxilla;
affixing an angularly adjustable maxillary distractor assembly on opposite sides of each said osteotomy; each said maxillary distractor assembly comprising a horizontal distractor assembly pivotally and detachably connected to a vertical arm member whereby the angle between said horizontal distractor assembly and said vertical arm member is adjustable, and a malar buttress plate member detachably connected to said vertical arm member; whereby said malar buttress plate member is affixed to the malar buttress and said horizontal distractor assembly is affixed to the maxilla;
performing the distraction process;
detaching said horizontal distractor assemblies from the maxilla and from said vertical arm members while said malar buttress plate members remain affixed to said malar buttress, and removing said horizontal distractor assemblies;
detaching said vertical arm members from said malar buttress plate members; and
leaving said malar buttress plate members affixed to the malar buttress and removing said vertical arm members, whereby said malar buttress plate members remain permanently affixed to said malar buttress.
